(19) United States
(12) Patent Application Publication
(10) Pub. No.: US 2025/0345623 A1
(43) Pub. Date: Nov. 13, 2025

(54) COSMETIC EYE APPLICATOR

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Tang Yuanzhou, Shenzhen (CN)

(73) Assignee: SHENZHEN NUON MEDICAL EQUIPMENT CO., LTD., Shenzhen (CN)

(21) Appl. No.: 19/196,790

(22) Filed: May 2, 2025

(30) Foreign Application Priority Data
May 7, 2024 (CN) 202420971516.X

(51) Int. Cl. A61N 5/06; A45D 34/04

(52) U.S. Cl. CPC A61N 5/0616; A45D 34/04; A45D 2200/155; A45D 2200/205; A61N 2005/0644; A61N 2005/0651

(57) ABSTRACT

Embodiments of the present invention provide a cosmetic applicator apparatus for a cosmetic eye applicator. The cosmetic applicator apparatus comprising a shell and at least one stimulation element configured to stimulate the skin. A liquid outlet channel runs through the shell, connecting to the bottle body. At the shell's output end, there are two distinct surfaces: an application surface and a stimulation surface, positioned on opposite sides. The application surface and the stimulation surface are oriented at intersecting angles, with the liquid outlet located on the application surface, and the stimulation component is placed near the stimulation surface. This structural arrangement ensures that the functions of liquid application and skin stimulation remain independent, preventing interference between the two.

### TECHNICAL FIELD

The present invention relates to an eye applicator for applying cosmetics, and more particularly, to a cosmetic applicator apparatus for applying cosmetics around the eyes.

### BACKGROUND

The appearance of wrinkles, fine lines, dark circles, and puffiness around the eyes are common cosmetic concerns associated with skin aging, particularly in delicate facial regions. In response, a variety of skin care products—such as eye creams and serums—are routinely applied to improve skin texture, reduce discoloration, and enhance the overall appearance of the periorbital area.

In recent advancements, cosmetic delivery systems have evolved beyond passive application, incorporating active stimulation technologies. Devices such as vibrators and phototherapy modules (e.g., LED light sources) have been integrated into cosmetic containers to enhance the penetration of skincare formulations and to promote microcirculation and cellular rejuvenation through physical or light-based stimulation.

However, integrating both a liquid outlet and a stimulation mechanism into the same output end of a cosmetic container introduces design challenges. Specifically, placing both the dispensing port and the stimulation component on a common end surface increases the footprint of the output area. This enlarged surface often reduces the applicator's ability to conform closely to the contours of the skin, thereby diminishing application precision, comfort, and treatment effectiveness.

To address these issues, there remains a need for an improved cosmetic applicator that can simultaneously deliver a skincare formulation and provide therapeutic stimulation, while maintaining a compact, ergonomic, and skin-conforming design. The present invention aims to fulfill this need by providing a cosmetic eye applicator in the form of a cosmetic applicator apparatus with a uniquely configured output end that separates the liquid dispensing function from the stimulation function, thereby enhancing both application efficiency and user experience.

### OBJECTS OF THE INVENTION

Some of the objects of the invention are as follows:

An object of the present invention is to provide a cosmetic applicator apparatus that enables both the application of a cosmetic liquid and the stimulation of the skin in a compact, functionally distinct, and user-friendly manner.

Another object of the invention is to physically separate the liquid application surfaces and the stimulation surface at the output end of the cosmetic applicator apparatus, thereby avoiding the interference between the cosmetic liquid and the stimulation function, and reducing the overall size of the output structure.

A further object of the invention is to provide a multi-functional stimulation element, which may include a phototherapy light source and/or a mechanical vibrator, to deliver non-invasive skin treatment while maintaining modular integration within the dispensing structure.

Another object of the invention is to enable the application and stimulation surfaces to conform ergonomically to the skin by adopting arc-shaped surface geometries, thereby enhancing user comfort and treatment effectiveness.

### SUMMARY OF THE INVENTION

According to a first aspect of the present invention, a cosmetic applicator apparatus is provided. The cosmetic applicator apparatus comprising: a shell having an output end and a connecting section, the output end comprises a first surface and a second surface located opposite to each other; at least one stimulation element is arranged on the first surface or the second surface for stimulating skin of a user; wherein the shell comprises a liquid outlet channel having a first end for communicating with a bottle body, and a second end that opens on the second surface of the output end of the shell; and wherein facing direction of the first surface and the second surface intersect at an angle.

In one embodiment of the invention, the at least one stimulation element is selected from a group consisting of a heating element, a cooling element, a vibration element, a Peltier element, a micro-current electrode, a phototherapy element, and an ultrasonic component.

In one embodiment of the invention, the at least one stimulation element comprises: a light source for performing light therapy on the skin, a circuit board electrically connected to the light therapy light source, and a battery electrically connected to the circuit board; wherein the light source is installed on the output end of the shell.

In one embodiment of the invention, the output end of the shell comprises a mounting groove for positioning the light source, and a light-transmitting cover is sealed on the mounting groove.

In one embodiment of the invention, the mounting groove comprises a positioning column connected to the light-transmitting cover, and the light source is sleeved on the positioning column.

In one embodiment of the invention, the shell comprises an output head for inserting into a cover body, and the connecting section comprises a bracket connected to the output head and a skirt shell sleeved outside the bracket; the bracket is provided with a first through hole, the output head is provided with a second through hole connected to the first through hole; wherein the first through hole and the second through hole forms the liquid outlet channel.

In one embodiment of the invention, the first surface and the second surface are arcuate surfaces to fit the skin.

In one embodiment of the invention, at least one of the first surface and the second surface is made with a flexible material to allow taking eye contour shape for efficient dispersal of a cosmetic.

In one embodiment of the invention, the second end of the liquid outlet channel further comprising: a heating element to pre-treat the oil before dispensing the oil over the eye area.

In one embodiment of the invention, the output head is flexible to rotate or tilt at an angle to allow a user to adjust the angle depending on the facial anatomy.

According to a second aspect of the present invention, a cosmetic applicator apparatus is provided. The cosmetic applicator apparatus comprising: a shell having an output end and a connecting end, the output end comprises a first surface and a second surface located opposite to each other; a first stimulation element arranged on the first surface for stimulating the skin; a second stimulation element arranged on the second surface for stimulating the skin; wherein the shell comprises a liquid outlet channel having a first end for communicating with a bottle body, and a second end that opens on the second surface of the output end of the shell; wherein facing direction of the first surface and the second surface intersect at an angle.

In one embodiment of the invention, the first stimulation element or the second stimulation element is selected from a group consisting of a heating element, a cooling element, a vibration element, a Peltier element, a micro-current electrode, a phototherapy element, and an ultrasonic component.

In one embodiment of the invention, the shell comprises an output head for inserting into a cover body.

In one embodiment of the invention, the connecting section comprises a bracket connected to the output head, and a skirt shell is sleeved outside the bracket.

In one embodiment of the invention, the shell is provided with a first electrode connected to a battery and the cover body is provided with a second electrode, the second electrode forms an electrical connection with the first electrode.

In one embodiment of the invention, the cover body comprises a charging interface to charge the battery when the first electrode is in electrical contact with the second electrode.

In one embodiment of the invention, the first surface and the second surface are inclined at an angle to form a V-shape.

In one embodiment of the invention, at least one of the first surface and the second surface is made with a flexible material to allow taking eye contour shape for efficient dispersal of a cosmetic.

In one embodiment of the invention, the second end of the liquid outlet channel further comprising: a heating element to pre-treat the oil before dispensing the oil over the eye area.

In one embodiment of the invention, the output head is flexible to rotate or tilt at an angle to allow a user to adjust the angle depending on the facial anatomy.

In the context of this specification, terms like “light”, “radiation”, “irradiation”, “emission” and “illumination”, etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for treatment of conditions that occur at the skin surface, such as psoriasis or infection.

In the context of the specification, the term “light source” or “phototherapy source” etc. refers to a source emitting coherent laser light, or light-emitting diodes (“LEDs”). The term “light therapy” refers to light generated from any of the sources, such as laser, LED sources, or Super luminous diodes (“SLD”).

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first’, “second” and “third” are only used for descriptive purposes and do not implicate the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, a “diaphanous material” is a material that allows at least a portion of one or more forms (such as Infrared, Ultraviolet, X-Rays, Visible Light, Microwaves, Radio Waves, etc.) of electromagnetic radiation to pass through them. The diaphanous materials can be transparent (allowing one or more forms of electromagnetic radiation to pass through with minimal scattering) or translucent (allowing one or more forms of electromagnetic radiation to pass through with appreciable diffusion or scattering). Diaphanous materials can be dense, like glass, or have an open structure, like wire mesh or a woven fabric.

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In the context of the specification, the term “cosmetic applicator apparatus” broadly refers to the assembly that facilitates both the dispensing of a liquid (eg. Skincare formulation, cosmetic) and the delivery of a skin stimulation function, typically mounted on or connected to a container bottle.

In the context of the specification, the term “Container bottle” or “bottle body” refers to any reservoir or housing structure that contains the liquid to be dispensed. It may include features such as an outlet opening for fluid communication with the liquid outlet mechanism.

In the context of the specification, the term “Shell” refers to the structural casing of the cosmetic applicator apparatus that houses internal components, defines the output end, and provides mechanical support for interfaces and features such as liquid channels, stimulation elements, or sealing covers.

In the context of the specification, the term “Application surface” refers to the portion of the output end of the shell through which the liquid is applied to the user's skin. This surface includes the outlet of the liquid outlet channel and is generally in direct contact with the skin during use.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 illustrates a top perspective view of a cosmetic applicator apparatus, in accordance with an embodiment of the present invention.

FIG. 2 illustrates a side view of the cosmetic applicator apparatus, in accordance with an embodiment of the present invention.

FIG. 3 illustrates a cross-sectional view of the cosmetic applicator apparatus head and a cover body, in accordance with an embodiment of the present invention.

FIG. 4 illustrates an exploded view of the cosmetic applicator apparatus head and the cover body, in accordance with an embodiment of the present invention.

FIG. 5 illustrates an exploded view of the cosmetic applicator apparatus head, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention disclose a dual-function cosmetic applicator apparatus. The cosmetic applicator apparatus is configured to both dispense a cosmetic liquid and deliver a skin stimulation effect while maintaining structural compactness and functional separation between dispensing and stimulation elements. The cosmetic applicator apparatus comprises a shell and a stimulation element configured to stimulate the skin. The shell is formed with a liquid outlet channel that communicates with a bottle body containing a cosmetic liquid or paste. At the output end of the shell, two distinct functional surfaces are provided, the first surface is a stimulation effect providing surface and the second surface is the application surface. The first surface and the second surface are located on opposite sides of the output end and are oriented such that their directions intersect, i.e. the first surface and the second surface face in different non-parallel directions. The outlet of the liquid outlet channel opens at the application surface allowing cosmetic liquid to be discharged and applied directly to the skin. The excitation component is disposed on the side of the shell adjacent to the first surface and is configured to deliver a stimulation effect through that surface.

The first surface and the second surface are arc-shaped. The curvature of these surfaces allows them to conform more naturally to the contours of the skin during use, thereby improving contact effectiveness and user comfort.

The cosmetic applicator apparatus may comprise one stimulation element on the first surface or more than one stimulation element is provided either on one surface or both the surface. The stimulation element is selected from a group consisting of one or more of a light-emitting component, a vibrating component, an ultrasonic generating component, an electrode component, a refrigeration component or a cooling component, a heating component, a Peltier component, and a magnetic component.

Several additional functionalities may also be added to the cosmetic applicator apparatus. For example, to enhance durability and optical efficiency, the apparatus includes a light-transmitting cover sealed over a mounting groove to protect the light source and facilitate the transmission of light to the skin. The user can convert one side of the first surface and the second surface facing the skin into the other side by rotating the shell 180 degrees thereby facilitating the user's control. The output end of the shell can also be set to a retractable structure, such as a lipstick-like retractable and extendable structure used between the output end and the shell. The cosmetic applicator apparatus includes a cover to cover and protect the output end. The cosmetic applicator apparatus comprises an input electrode arranged on the output head and is electrically connected to the battery, and the cover is provided with an output electrode connected to a charging interface on the cover. When the cover is applied over the apparatus, the input electrode connects with the output electrode, and charging of the battery is realized by connecting the cover to a charger.

Several embodiments of the present invention will now be described in detail with references to FIGS.

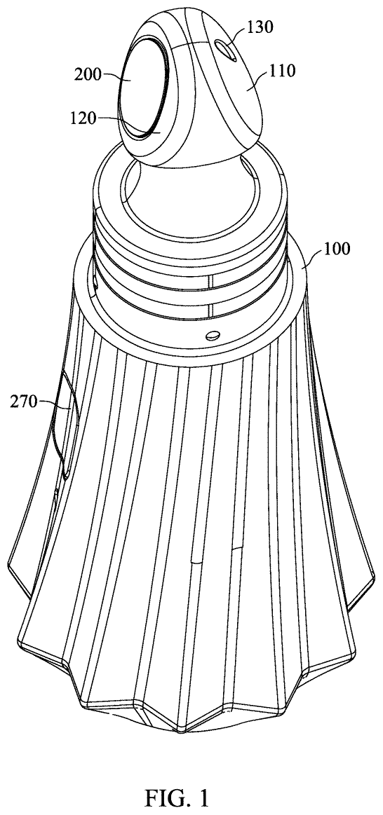

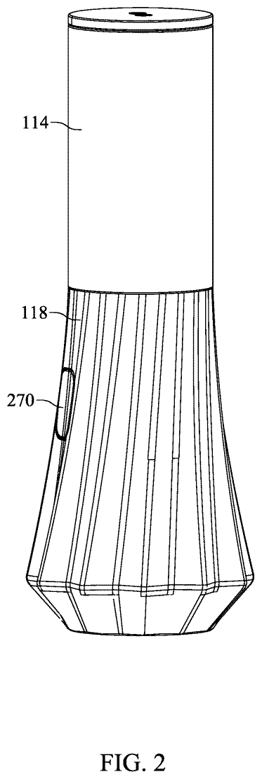

Referring to FIG. 1 and FIG. 2, a cosmetic applicator apparatus that can be used as a cosmetic eye applicator for applying cosmetic liquid, such as an eye cream applicator with a bottle is illustrated. The cosmetic liquid herein refers to a liquid substance or a paste substance that can be dispensed through the cosmetic applicator apparatus. The cosmetic applicator apparatus provided in the embodiments of the present application can be connected to a bottle body storing cosmetic coating liquid, such as an eye cream bottle or container.

The cosmetic applicator apparatus comprises a shell 100 and at least one stimulation element 200 for stimulating the skin. The shell 100 has an output end, provided with two opposite surfaces: a first surface 120 and a second surface 110. The first surface 120 acts as a surface for providing a stimulation effect by the at least one stimulation element 200, and the second surface 110 acts as an applicator surface for applying cosmetics. The facing direction of the first surface 120 intersects with the facing direction of the second surface 110. A liquid outlet channel 130 is provided in the shell 100 and when the shell 100 connects the bottle body, a first end of the liquid outlet channel connects to the inside of the bottle body. A second end of the liquid outlet channel 130 is provided on the second surface 110. The liquid outlet channel 130 passes through and opens on the second surface 110 so that the application liquid/cosmetic component is dispensed out from the second surface 110. The stimulation element 200 is arranged on the output end of the shell 100 close to the first surface 120. The stimulation element 200 excites the skin and outputs the stimulation energy through the first surface 120.

In an embodiment of the present invention, the first surface and the second surface are inclined at an angle to form a V-shape.

In an embodiment of the present invention, the first surface 120 and the second surface 110 converge at a point to form a tip or an edge. The tip or the edge is flexible so that it will not hinder the cosmetic application process. The flexible material can be a LVT material, a VCT material, a rubber material, a gel material, or a similar material.

In an embodiment of the present invention, the liquid outlet channel 130 opens on the second surface and the cosmetic liquid is dispensed through a dispenser tip. The dispenser tip includes a tiny heating clement arranged around the dispenser tip to slightly warm the oil/cosmetic (25° C. to 40° C.) before it touches the eye area.

The second surface 110 and the first surface 120 can be a plane or an arcuate surface with a small curvature. When the second surface 110 and the first surface 120 are planes, the orientation of the two refers to the normal direction of the two. The orientation of the second surface 110 refers to the direction perpendicular to the normal of the second surface 110, and the orientation of the first surface 120 refers to the direction perpendicular to the normal of the first surface 120. When the second surface 110 and the first surface 120 are arc-shaped concave surfaces, the orientation of the second surface 110 and the first surface 120 refers to the opening direction of the second surface 110 and the first surface 120. When the second surface 110 and the first surface 120 are arc-shaped convex surfaces, the orientation of the second surface 110 and the first surface 120 refers to the convex direction of the second surface 110 and the first surface 120.

In an embodiment of the present invention, the first surface or the second surface or both the first surface and the second surface are made of flexible material so that during application, the surface conforms to the eye contour shape allowing more efficient dispensing of the oil/cosmetic. The flexible material can be a LVT material, a VCT material, a rubber material, a gel material, or a similar material.

The liquid outlet channel 130 and the stimulation element 200 are respectively arranged on the side of the second surface 110 and the shell 100 close to the first surface 120, so that the second surface 110 and the first surface 120 can have a smaller area. At the same time, the direction of the second surface 110 and the direction of the first surface 120 intersect, so that the first surface 120 and the second surface 110 can form an angle between the two, thereby reducing the end surface area on the output end of the shell 100 and reducing the overall volume of the output end of the shell 100, and avoiding the mutual interference between the smearing liquid and the stimulation treatment.

In an embodiment, the second surface 110 and the first surface 120 can be two surfaces that may or may not be smoothly connected structures, such as the output end of the shell 100 to a curved surface structure protruding outside, etc. Specifically, the outlet direction of the liquid outlet channel 130 and the output direction of the stimulation element 200 are respectively oriented in different directions to achieve the effect of reducing the end surface area of the output end of the shell 100.

In an embodiment, the second surface 110 and the first surface 120 are respectively arranged on opposite sides of the output end of the shell 100. Taking the angle of the cosmetic applicator apparatus shown in FIG. 1 as an example, the second surface 110 can be arranged on the right side of the shell 100, and the first surface 120 can be arranged on the left side of the shell 100.

In an embodiment of the present invention, the output end of the cosmetic applicator apparatus is configured to be rotatable and bendable on the horizontal axis, allowing a user to adjust the angle depending on their facial structure.

The user can convert one side of the second surface 110 and the first surface 120 facing the skin into the other side by rotating the shell 100 by 180° while using, thereby facilitating the user's control. The second surface 110 and the first surface 120 are respectively arranged on opposite sides of the shell 100 to help reduce the volume of the output end of the shell 100.

In an embodiment, the second surface 110 and/or the first surface 120 are arc-shaped surfaces so that the second surface 110 and/or the first surface 120 can fit the skin. The second surface 110 and/or the first surface 120 may be, but are not limited to, an arc-shaped convex surface, and may be adjusted according to the application location of the liquid outlet mechanism so that the second surface 110 and/or the first surface 120 can better fit the user's skin. The output end of the shell 100 may be configured as a structure located outside the shell 100 so that the cosmetic applicator apparatus can be used directly.

In another embodiment, the output end of the shell 100 can also be set to a retractable structure, for example, a lipstick-like retractable and extendable structure is used between the output end of the shell 100 and the shell 100, and the output end of the shell 100 can be controlled to extend outside the shell 100 while using.

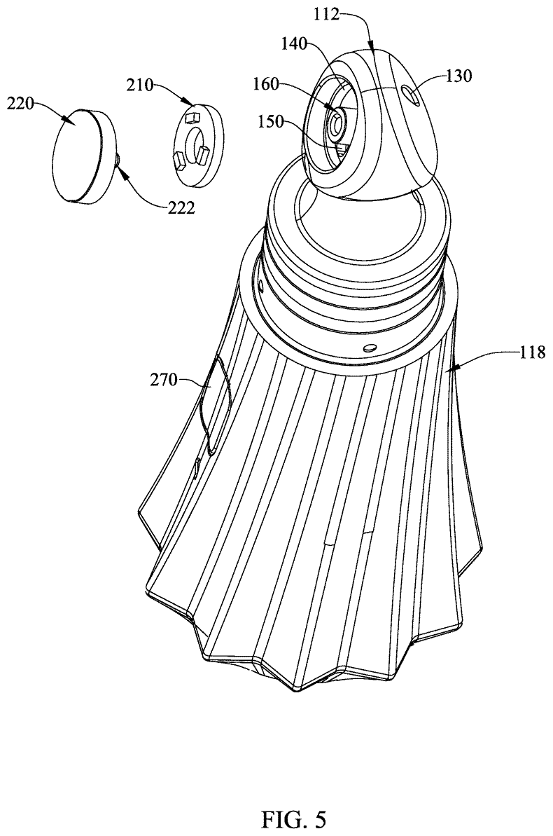

Referring to FIG. 2 and FIG. 5, in an embodiment of the present invention, the shell 100 may include a cover body 114 to cover and protect the output end of the shell 100.

In an embodiment of the present invention, the stimulation element 200 may include: one or more of a light-emitting component, a vibrating component, an ultrasonic generating component, an electrode component, a refrigeration component, or a cooling component, a heating component, a Peltier component, and a magnetic component.

Referring to FIG. 1 to FIG. 3, the stimulation element 200 in the form of a phototherapy light source 210 integrated with the cosmetic applicator apparatus is illustrated. The phototherapy light source 210 is arranged at one end of the output end of the shell 100 close to the first surface 120. The phototherapy light source 210 is used to emit light that can pass through the first surface 120 and can promote the absorption of the coating liquid and/or perform phototherapy on the skin.

The phototherapy light source 210, which may be implemented as an LED light board, is configured to perform phototherapy on the skin. By positioning the phototherapy light source 210 adjacent to the first surface 120, the phototherapy function is spatially separated from the skincare liquid application, allowing both operations to occur independently without mutual interference.

Referring to FIG. 5, the output end of the shell 100 is provided with a mounting groove 140 for installing the phototherapy light source 210. The mounting groove 140 can be provided on the inner side of the first surface 120. The phototherapy light source 210 is arranged in the mounting groove 140. Furthermore, the stimulation element 200 is also equipped with a light-transmitting cover 220 and the light-transmitting cover 220 is sealed on the mounting groove 140. The light transmitting cover 220 can be made of diaphanous material.

The light-transmitting cover 220 can allow the light emitted by the phototherapy light source 210 to pass through, playing a light-guiding role. A wire hole 150 connected to the bottom of the mounting groove 140 is provided in the output end of the shell 100. The wire hole 150 is used for the connection wire of the phototherapy light source 210 to pass through so that the connection wire of the phototherapy light source 210 can be electrically connected to the circuit board 260.

In an embodiment of the present invention, the stimulation element 200 is electrically connected to a circuit board 260 and a battery 250. The circuit board 260 and the battery 250 are arranged in the shell 100, the battery 250 is electrically connected to the circuit board 260 and is used to power the circuit board 260. The circuit board 260 is electrically connected to the phototherapy light source 210 or the stimulation element 200 and is used to control the start stop and lighting mode of the phototherapy light source 210. Correspondingly, the stimulation element 200 may include a control button 270 which is connected to the circuit board 260 and extends out of the shell 100, so that the user can control the stimulation element 200 through the control button 270 and the circuit board 260.

In an embodiment of the present invention, the cosmetic applicator apparatus comprises two or more stimulation elements. A first stimulation element is positioned on the first surface and a second stimulation element is positioned on the second surface. In an alternate configuration, the first stimulation element and the second stimulation element are arranged either on the first surface or the second surface.

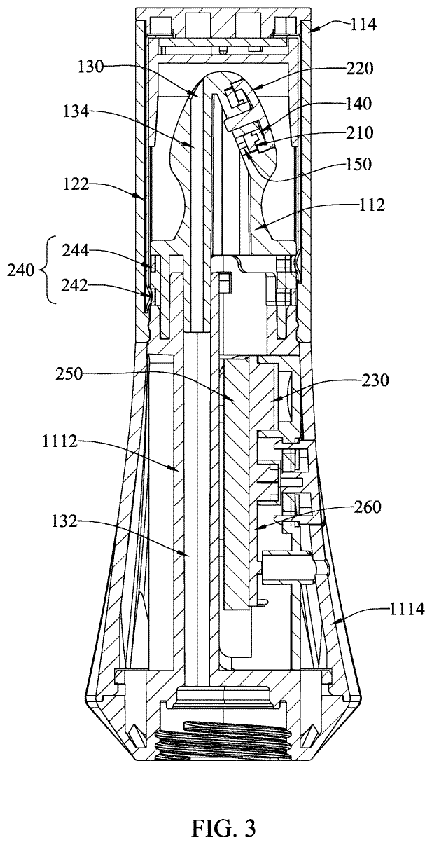

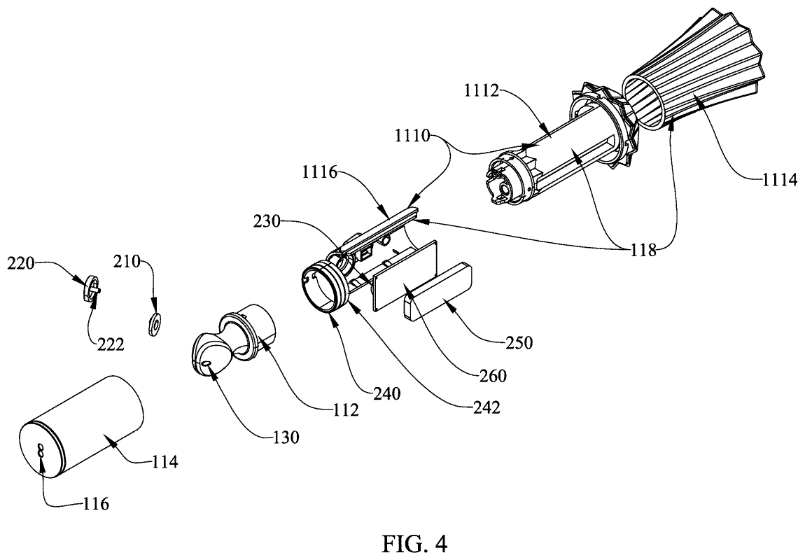

Referring to FIG. 3 to FIG. 5, a positioning column 160 is provided within the mounting groove 140 and is connected to the light-transmitting cover 220. The positioning column 160 may be fixed to the light-transmitting cover 220, such that when the cover is mounted onto the mounting groove 140, the column inserts into the groove and engages with the shell 100. For example, the positioning column 160 may be securely fixed within the mounting groove 140, allowing the phototherapy light source 210 to be sleeved over the column for stable positioning. The light-transmitting cover 220 is then mounted onto the groove and connected to the positioning column 160, ensuring proper alignment and secure assembly.

The positioning column 160 is disposed within the mounting groove 140 and includes a central through hole. The light-transmitting cover 220 is provided with a connecting column 222 at its center. During assembly, the connecting column 222 is inserted into the through hole of the positioning column 160. This configuration serves as a guiding mechanism to facilitate accurate and secure installation of the light-transmitting cover 220.

In an embodiment, the stimulation element 200 in the cosmetic applicator apparatus is a vibrator 230 positioned within the shell 100 and mounted on the circuit board 260. The vibrator 230 generates vibrations that are transmitted through the shell 100, causing the first surface 120 to massage the skin and help relax underlying muscles.

In an embodiment of the present invention, the shell 100 comprises a connecting section 118 and an output head 112. The connecting section 118 is configured to attach to the bottle body, while the output head 112 is connected to the connecting section 118 and is insertable into the cover body 114. The second surface 110 and the first surface 120 are positioned on opposite sides of the output head 112. The circuit board 260 and battery 250 are housed within the connecting section 118. The output head 112 functions as the output end of the shell 100.

The connecting section 118 includes a skirt shell 1114 and a bracket 1110. The bracket 1110 connects to the output head 112, and the skirt shell 1114 is fitted over the bracket 1110. The bracket 1110 is formed with a first through hole 134, while the output head 112 includes a second through hole 132 that aligns with the first through hole 134. Together, the first and second through holes form the liquid outlet channel 130.

In order to facilitate the installation of the circuit board 260 and the battery 250, the bracket 1110 may include an inner frame 1112 and an inner shell 1116. The components such as the circuit board 260, the battery 250, and the vibrator 230 may be assembled on the inner shell 1116. The second through hole 132 is provided on the inner frame 1112, and then the inner frame 1112 and the inner shell 1116 are combined to form the bracket 1110 of the connecting section 118.

The cross-sectional area of the connecting section 118 tapers toward the output head 112. Specifically, the skirt shell 1114 features a waist-like design, with its cross-section narrowing in the direction of the output head 112. This tapered structure helps minimize visual obstruction for the user during application and reduces contact with the nose or other facial areas. Additionally, the outer surface of the skirt shell 1114 is formed with multiple pleated features, enhancing both the aesthetic appeal and the grip of the connecting section 118.

The cover body 114 is detachably connected to the connecting section 118. When the cover body 114 is attached, it encloses the output head 112, positioning it within the interior of the cover.

In one embodiment of the present invention, the cosmetic applicator apparatus includes an input electrode 240 positioned on the output head 112. The input electrode 240 is electrically connected to the battery 250. When connected to an external power source, the input electrode 240 enables the charging of the battery 250. The input electrode 240 may comprise a first electrode 244 and a second electrode 242, which function as the positive and negative terminals, respectively.

A charging interface 116 is provided on the cover body 114. Inside the cover, an output electrode 122 is electrically connected to the charging interface 116. The input electrode 240, located on the exterior of the output head 112, is also electrically connected to the battery 250. When the cover body 114 is attached to the connecting section 118, the output electrode 122 comes into electrical contact with the input electrode 240, allowing for battery charging.

The charging interface 116 on the cover body 114 can be connected to an external charger, enabling the output electrode 122 to supply power to the input electrode 240. In this configuration, charging can only occur when the cover body 114 is attached to the connecting section 118. This ensures that the liquid outlet mechanism is inactive during charging, preventing the output head 112 from contacting the user's skin and thereby improving safety.

Additionally, the output electrode 122 and/or the input electrode 240 can be arranged in a surrounding configuration—such as a ring shape. This design allows the cover body 114 to connect to the connecting section 118 from any orientation while still ensuring electrical contact between the electrodes, thereby enhancing case of use.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A cosmetic applicator apparatus comprising:
a shell having an output end and a connecting section, the output end comprises a first surface and a second surface located opposite to each other;
at least one stimulation element arranged on the first surface or the second surface for stimulating skin of a user;
wherein the shell comprises a liquid outlet channel having a first end for communicating with a bottle body, and a second end that opens on the second surface of the output end of the shell; and
wherein facing direction of the first surface and the second surface intersect at an angle.

2. The cosmetic applicator apparatus of claim 1, wherein the at least one stimulation element is selected from a group consisting of a heating element, a cooling element, a vibration element, a Peltier element, a micro-current electrode, a phototherapy element, and an ultrasonic component.

3. The cosmetic applicator apparatus of claim 1, wherein the at least one stimulation element comprises: a light source for performing light therapy, a circuit board electrically connected to the light therapy light source, a battery electrically connected to the circuit board; and wherein the light source is installed on the output end of the shell.

4. The cosmetic applicator apparatus of claim 3, wherein the output end of the shell comprises a mounting groove for positioning the light source, and a light-transmitting cover is sealed on the mounting groove.

5. The cosmetic applicator apparatus of claim 4, wherein the mounting groove comprises a positioning column connected to the light-transmitting cover and the light source is sleeved on the positioning column.

6. The cosmetic applicator apparatus as claimed in claim 1, wherein the shell comprises an output head for inserting into a cover body, and the connecting section comprises a bracket connected to the output head and a skirt shell sleeved outside the bracket; the bracket is provided with a first through hole, the output head is provided with a second through hole connected to the first through hole; and wherein the first through hole and the second through hole forms the liquid outlet channel.

7. The cosmetic applicator apparatus of claim 1, wherein the first surface and the second surface are arcuate surfaces to fit the contours of the user.

8. The cosmetic applicator apparatus of claim 1, wherein at least one of the first surface and the second surface is made with a flexible material to allow taking eye contour shape for efficient dispersal of a cosmetic.

9. The cosmetic applicator apparatus of claim 1, wherein the second end of the liquid outlet channel further comprising: a heating element to pre-treat a cosmetic before dispensing on the cosmetic over the eyes area.

10. The cosmetic applicator apparatus of claim 1, wherein the output end is flexible to rotate or tilt at an angle to allow a user to adjust the angle depending on facial anatomy of the user.

11. A cosmetic applicator apparatus comprising:
a shell having an output end and a connecting section, the output end comprises a first surface and a second surface located opposite to each other;
a first stimulation element arranged on the first surface for stimulating skin of a user;
a second stimulation element arranged on the second surface for stimulating skin of the user;
wherein the shell comprises a liquid outlet channel having a first end for communicating with a bottle body, and a second end that opens on the second surface of the output end of the shell; and
wherein facing direction of the first surface and the second surface intersect at an angle.

12. The cosmetic applicator apparatus of claim 11, wherein the first stimulation element or the second stimulation element is selected from a group consisting of a heating element, a cooling element, a vibration element, a Peltier element, a micro-current electrode, a phototherapy element, and an ultrasonic component.

13. The cosmetic applicator apparatus of claim 11, wherein the shell comprises an output head for inserting into a cover body.

14. The cosmetic applicator of claim 13, wherein the connecting section comprises a bracket connected to the output head, and a skirt shell is sleeved outside the bracket.

15. The cosmetic applicator apparatus of claim 13, wherein the shell is provided with a first electrode connected to a battery and the cover body is provided with a second electrode, the second electrode forms an electrical connection with the first electrode.

16. The cosmetic applicator apparatus of claim 14, wherein the cover body comprises a charging interface to charge the battery when the first electrode is in electrical contact with the second electrode.

17. The cosmetic applicator apparatus of claim 11, wherein the first surface and the second surface are inclined at an angle to form a V-shape.

18. The cosmetic applicator apparatus of claim 11, wherein at least one of the first surface and the second surface is made with a flexible material to allow taking eye contour shape for efficient dispersal of a cosmetic.

19. The cosmetic applicator apparatus of claim 11, wherein the second end of the liquid outlet channel further comprising: a heating element to pre-treat a cosmetic before dispensing on the cosmetic over eyes area.

20. The cosmetic applicator apparatus of claim 11, wherein the output end is flexible to rotate or tilt at an angle to allow a user to adjust the angle depending on facial anatomy of the user.
